(19) United States
(12) Patent Application Publication
(10) Pub. No.: US 2018/0116459 A1
(43) Pub. Date: May 3, 2018

(54) FRYING APPARATUS CAPABLE OF REAL-TIMELY DETECTING TOTAL POLAR MATERIAL CONCENTRATION OF OIL

(71) Applicant: TECO Electric & Machinery Co., Ltd., Taipei City (TW)

(72) Inventors: Chia-Jen LIN, Taipei City (TW); Feng-Chieh LIN, Taipei City (TW)

(21) Appl. No.: 15/375,715

(22) Filed: Dec. 12, 2016

(30) Foreign Application Priority Data
Oct. 28, 2016 (TW) 105135113

(51) Int. Cl. A47J 37/12; G01N 33/28

(52) U.S. Cl. CPC A47J 37/1271; G01N 33/2835; A47J 37/1276

(57) ABSTRACT

A frying apparatus includes a main body, a frying vat, a fry basket and a detection module. The frying vat located on the main body has a first polar plate. The fry basket includes a basket frame and a second polar plate. When the fry basket is positioned inside the frying vat, the second polar plate is parallel to and separated from the first polar plate to form an equivalent capacitor. The detection module is electrically connected with the first and second polar plates. When the first and second polar plates are dipped in the oil contained inside the frying vat, the detection module detects a capacitance of the equivalent capacitor, then a corresponding dielectric constant of the oil is obtained by evaluating the capacitance and at least one coupling condition, and further a corresponding total polar material concentration of the oil with respect to the dielectric constant can be deduced.

This application claims the benefit of Taiwan Patent Application Serial No. 105135113, filed Oct. 28, 2016, the subject matter of which is incorporated herein by reference.

### BACKGROUND OF INVENTION

### 1. Field of the Invention

The invention relates to a frying apparatus, and more particularly to the frying apparatus that can detect the total polar material concentration of the contained oil therein in a real-time manner.

### 2. Description of the Prior Art

In the era of great advance in modern technology, quality of life has been continuously improved, with the even rising demands in better lifestyles. In particular, people have paid more attention on the health issues and the safety of food intake. For example, in Taiwan, after experiencing several major food safety events in this decade, food knowledge regarding personal health has become popular. Especially, safety knowledge of the everyday's cooking oil has become one of crucial issues to people's concern.

After being heated up to a specific high temperature, cooking oils would usually generate oxides, free fatty acids, total polar compounds, and the like harmful substances. Thus, in most of countries in this planet, regulations and standards are legislated to specify the legal contents of oxides, free fatty acids, total polar compounds and the like substances in the edible or cooking oils. In particular, by having Taiwan's food regulations for example, the legal content of total polar compounds in the cooking oil shall not exceed 25 wt %.

In order for the oil products to meet the regulations and standards, various methods and instruments have been developed to detect the content of the total polar material in the oil. In general, the well-known column chromatography is applied to detect the content of the total polar material. To perform the column chromatography, analysis of oil samples shall be performed in the laboratory by professional instruments, and such an analysis usually takes about 6 hours. Namely, the column chromatography, though popular, is pretty time-consumed. Alternatively, the PTC total polar test papers can also be conveniently applied to detect the content of the total polar material, but needs 30˜40 minutes at least for the testing results. In addition, the aforesaid two methods need naked eyes to judge the results, and thus possible visual error is inevitable.

In addition, another instrument for detecting the cooking oil and its compounds can be applied to detect directly the total polar material in the cooking oil, and the testing time needs only minutes. However, such an instrument is too expensive to be afforded for most of the practitioners. Although the testing time required by this instrument is much acceptable by compared with the aforesaid two methods, yet the testing time is still room for improvement.

As stated above, if the column chromatography or the PTC total polar test papers are applied to detect the content of the total polar material in the cooking oil, detection errors and longer testing time are inevitable. On the other hand, if the instrument for detecting the cooking oil and its compounds is applied to detect the content of the total polar material in the cooking oil, though the testing time can be greatly reduced, yet the equipment expense is a problem.

### SUMMARY OF THE INVENTION

In view of the aforesaid prior art for detecting the content of the total polar material in the cooking oil, the column chromatography and the PTC total polar test papers do have disadvantages in poor detection accuracy and longer testing time, and, on the other hand, the instrument for detecting the cooking oil and its compounds has the disadvantage in unacceptable equipment cost.

Accordingly, it is the primary object of the present invention to provide a frying apparatus capable of real-timely detecting a total polar material concentration of an oil. The frying apparatus includes a main body, a frying vat, a fry basket and a detection module. The frying vat for containing the oil, especially a cooking or edible oil, is located on the main body, and further includes thereinside a first polar plate.

The fry basket includes a basket frame and a second polar plate. The basket frame is to be placed into the frying vat. The second polar plate is connected with the basket frame. When the fry basket is positioned inside the frying vat, the second polar plate is parallel to and separated from the first polar plate so as to form an effective region for formulating a parallel capacitance effect between the first polar plate and the second polar plate. The first polar plate and the second polar plate fulfill at least one coupling condition.

The detection module is electrically connected with the first polar plate and the second polar plate. When the first polar plate and the second polar plate are dipped in the oil contained inside the frying vat so as to have the oil to fill the effective region, the detection module detects an equivalent capacitance induced between the first polar plate and the second polar plate, a corresponding dielectric constant of the oil is then obtained by evaluating the equivalent capacitance and the at least one coupling condition, and further a corresponding total polar material concentration of the oil with respect to the dielectric constant is deduced.

In one embodiment of the present invention, the main body includes a first conductive connector electrically connected with the detection module, and the fry basket further includes a second conductive connector located at the basket frame, electrically connected with the second polar plate, and connected with the first conductive connector in a removable manner.

In one embodiment of the present invention, an electric wiring is further included to bridge electrically the second conductive connector and the second polar plate. Generally, electric connection between the second conductive connector and the second polar plate can be served by the fry basket made of a conductive material. However, the electric connection between the second conductive connector and the second polar plate through the electric wiring can provide better conduction effect than that through the fry basket.

In one embodiment of the present invention, the first conductive connector is a conductive rod and the second conductive connector is a conductive hook.

In one embodiment of the present invention, the detection module is located at the main body.

In one embodiment of the present invention, the detection module further includes an oscillation-frequency generation unit and a frequency analysis unit. The oscillation-frequency generation unit electrically connected with each of the first polar plate and the second polar plate is to generate an oscillation signal when an equivalent capacitor in the effective region filled with the oil is formed by the first polar plate and the second polar plate. Then, the oscillation signal is further analyzed to obtain a corresponding oscillation frequency of the oil. The frequency analysis unit electrically connected with the oscillation-frequency generation unit has frequency-concentration reference information. When the at least one coupling condition is satisfied, the oscillation-frequency generation unit bases on the oscillation frequency to inquire the corresponding total polar material concentration of the oil from the frequency-concentration reference information.

In one embodiment of the present invention, the first polar plate is one of an inner wall of the frying vat or a conductive plate.

In one embodiment of the present invention, the frying apparatus further includes a heating member for heating the oil in the frying vat.

In one embodiment of the present invention, the frying apparatus further includes an alert module for alerting a message related to the total polar material concentration. The alert module is communicatively connected with the detection module.

In one embodiment of the present invention, the fry basket further includes a handle structure connected structurally with the basket frame. When the basket frame is positioned in the frying vat, the handle structure is located out of the frying vat from the basket frame.

In another aspect of the present invention, the frying apparatus capable of real-timely detecting a total polar material concentration of an oil includes a main body, a frying vat, a second polar plate and a detection module. The frying vat for containing an oil is located on the main body and further includes a first polar plate. The second polar plate is located in the frying vat at a position corresponding to the first polar plate so as to form an effective region for formulating a parallel capacitance effect between the first polar plate and the second polar plate. The first polar plate and the second polar plate fulfill at least one coupling condition. The detection module is electrically connected with the first polar plate and the second polar plate.

When the first polar plate and the second polar plate are dipped in the oil contained inside the frying vat so as to have the oil to fill the effective region, the detection module detects an equivalent capacitance induced between the first polar plate and the second polar plate, a corresponding dielectric constant of the oil is then obtained by evaluating the equivalent capacitance and the at least one coupling condition, and further a corresponding total polar material concentration of the oil with respect to the dielectric constant is deduced.

In one embodiment of the present invention, the second polar plate is electrically connected the detection module via a polar-plate connection arm extending out of the frying vat from the second polar plate.

By providing the pair of the first polar plate and the second polar plate to the frying apparatus in accordance with the present invention, an equivalent capacitor in the effective region for formulating the parallel capacitance effect can be formed in the oil contained in the frying vat. Thereupon, the detection module can be applied to detect the equivalent capacitance of the equivalent capacitor. Based on the detected equivalent capacitance, a corresponding dielectric constant of the oil can be calculated. Then, the oscillation-frequency generation unit is applied to transform the dielectric constant into a corresponding oscillation frequency of the oil. Finally, by referring to the frequency-concentration reference information, a corresponding total polar material concentration of the oil can be inquired.

By compared with the prior art, the frying apparatus of the present invention can transform the detected equivalent capacitance into the total polar material concentration via the detection module in a real-time manner. In comparison with the column chromatography and the PTC total polar test papers in the art, the detection function provided by the frying apparatus of the present invention is much more efficient and accurate. Further, since the frying apparatus of the present invention can measure the total polar material concentration of the oil in a real-time manner, thus a huge expense to purchase additional instruments for detecting the total polar material concentration of the oil can be saved.

All these objects are achieved by the frying apparatus capable of real-timely detecting a total polar material concentration of an oil described below.

### BRIEF DESCRIPTION OF THE DRAWINGS

The present invention will now be specified with reference to its preferred embodiment illustrated in the drawings, in which:

FIG. 1 is a schematic perspective view of a first embodiment of the frying apparatus capable of real-timely detecting a total polar material concentration of an oil in accordance with the present invention;

FIG. 2 is a schematic side view of FIG. 1;

FIG. 3 is an application of FIG. 2;

FIG. 4 is an enlarged view of area A of FIG. 3;

FIG. 5 is a schematic circuit view of the first embodiment of the frying apparatus capable of real-timely detecting a total polar material concentration of an oil in accordance with the present invention;

FIG. 6 is a schematic block view of the first embodiment of the frying apparatus capable of real-timely detecting a total polar material concentration of an oil in accordance with the present invention;

FIG. 7 is a flowchart of a preferred detection method for the first embodiment of the frying apparatus to undergo a real-time detection of the total polar material concentration of a cooking oil in accordance with the present invention;

FIG. 8 is a schematic side view of a second embodiment of the frying apparatus capable of real-timely detecting a total polar material concentration of an oil in accordance with the present invention;

FIG. 9 is an application of FIG. 8;

FIG. 10 is an enlarged view of area B of FIG. 9;

FIG. 11 is a schematic perspective view of a third embodiment of the frying apparatus capable of real-timely detecting a total polar material concentration of an oil in accordance with the present invention, with the fry basket separated; and

FIG. 12 is a schematic side view of FIG. 11, without the fry basket, but with the cooking oil.

### DESCRIPTION OF THE PREFERRED EMBODIMENT

The invention disclosed herein is directed to a frying apparatus capable of real-timely detecting a total polar material concentration of an oil. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

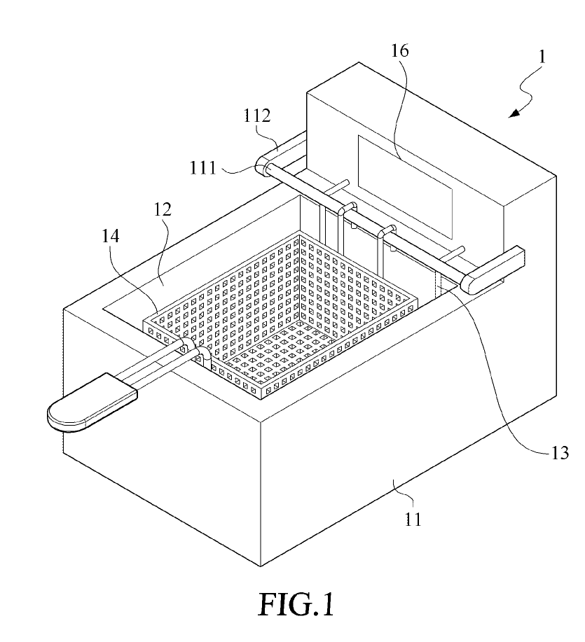

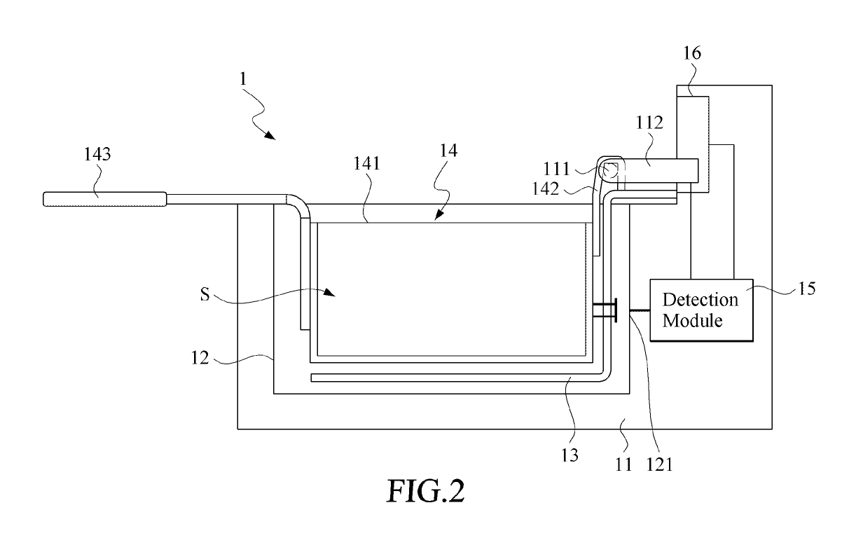

Refer now to FIG. 1 and FIG. 2; where FIG. 1 is a schematic perspective view of a first embodiment of the frying apparatus capable of real-timely detecting a total polar material concentration of an oil in accordance with the present invention, and FIG. 2 is a schematic side view of FIG. 1. As shown, the first embodiment 1 of the frying apparatus capable of real-timely detecting a total polar material concentration of an oil (especially, a cooking or edible oil) includes a main body 11, a frying vat 12, a heating member 13, a fry basket 14, a detection module 15 and an alert module 16.

The detection module 15 is located at the main body 11. A first conductive connector 111, formed as a horizontal bar supported structurally by two connection arms 112 extending outward from an upper portion of the main body 11, is electrically coupled with the detection module 15, but is electrically insulated to a housing of the main body 11. In details, two opposing ends of the first conductive connector 111 are individually held by corresponding free ends of the two connection arms 112 (one labeled in the figure), while other ends of the two connection arms 112 are connected structurally to the housing of the main body 11. Definitely, the first conductive connector 111 is electrically insulated to the connection arms 112. In this embodiment, the first conductive connector 111 is, but not limited to, a conductive rod.

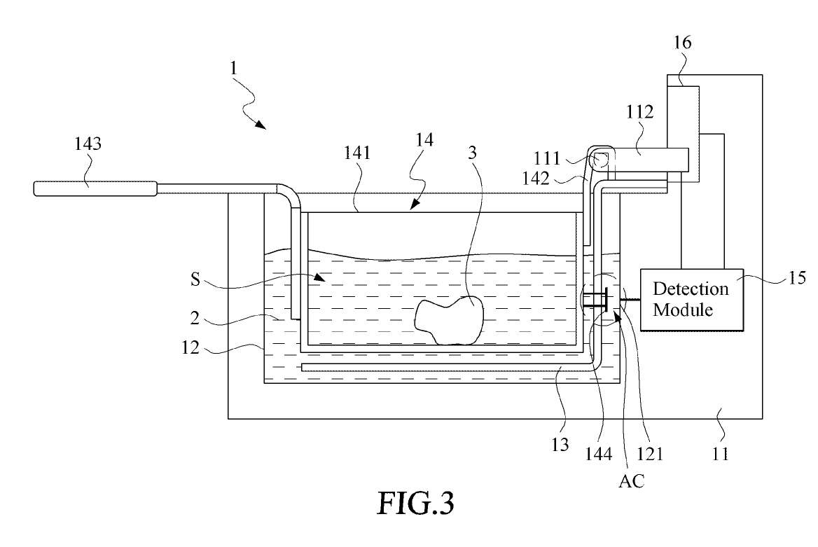

Referring now to FIG. 3, an application of the first embodiment of FIG. 2 is shown. The frying vat 12 for containing the oil 2 for frying is located on the main body 11, and further includes a first polar plate 121. In this embodiment, the first polar plate 121 is formed by, but not limited to, an inner wall of the frying vat 12 or a portion of the inner wall. The heating member 13 is extended from a portion of the main body 11 outside the frying vat 12 to a lower portion inside the frying vat 12. The heating member 13 is to heat up the oil 2 in the frying vat 12 so as to fry an object dipped in the oil 2. While the heating member 13 is heating the oil 2, a total polar material concentration of the oil 2 would gradually go up.

The fry basket 14 includes a basket frame 141, a second conductive connector 142, a handle structure 143 and a second polar plate 144. The basket frame 141 to be placed into the frying vat 12 provides therein a frying space S for disposing at least one object 3 to be fried. The basket frame 141 is formed as a net structure for providing a plurality of holes to allow the oil 2 to flow freely in and out of the frying space S as well as the basket frame 141.

The second conductive connector 142 connected with the basket frame 141 is to connect with the first conductive connector 111 in a removable manner. In this embodiment, the second conductive connector 142 is formed as a conductive hook to hook onto the conductive rod (i.e. the first conductive connector 111). The handle structure 143 is connected with the basket frame 141. While the basket frame 141 is disposed inside the frying vat 12, the handle structure 143 would be located out of the frying vat 12, so that a user can hold the handle structure 143 to operate the fry basket 14 without touching the oil in the frying vat 12.

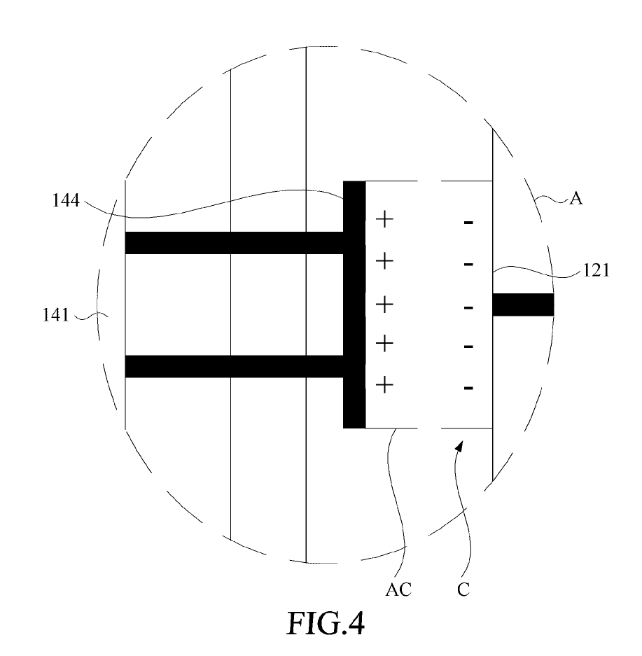

Refer now to both FIG. 3 and FIG. 4, where FIG. 4 is an enlarged view of area A of FIG. 3. As shown, the second polar plate 144 is mounted at the basket frame 141 and electrically connected with the second conductive connector 142 through the basket frame 141, in which the basket frame 141 is made of a conductive material. While the fry basket 14 is positioned inside the frying vat 12, the second polar plate 144 would be parallel to and separated from the first polar plate 121 by a predetermined gap, such that the first polar plate 121 and the second polar plate 144 can be integrated to act as an effective region AC for formulating the parallel capacitance effect.

In the present invention, to have the pair of the first polar plate 121 and the second polar plate 144 to perform an effective capacitor, at least one coupling condition shall be fulfilled. The at least one coupling condition shall include satisfied ranges or parameters for spacing between the first polar plate 121 and the second polar plate 144, an effective area for the second polar plate 144 to match the first polar plate 121 (or the first polar plate 121 to match the second polar plate 144), and conductivity of the first polar plate 121 and the second polar plate 144. When the fry basket 14 is positioned inside the frying vat 12 so as to have the second polar plate 144 to be energized and to face the first polar plate 121, and if and only if the pair of the first polar plate 121 and the second polar plate 144 satisfy all the at least one coupling condition, then an equivalent capacitor C can be formed by the first polar plate 121 and the second polar plate 144.

The detection module 15 located at the main body 11 is electrically connected to each of the first polar plate 121 and the second polar plate 144. The detection module 15 is coupled electrically and distantly to the second polar plate 144 orderly through the first conductive connector 111, the second conductive connector 142 and the basket frame 141. As the first polar plate 121 and the second polar plate 144 are both dipped in the oil 2 in the frying vat 12, the oil 2 will also fill the effective region AC for formulating the parallel capacitance effect, such that the equivalent capacitor is formed. Namely, an equivalent capacitance value between the first polar plate 121 and the second polar plate 144 is detected by the detection module 15. In addition, based on the equivalent capacitance value and the at least one coupling condition to compute a corresponding dielectric constant of the oil 2. Further, based on the computed dielectric constant, a corresponding total polar material concentration of the oil 2 can be deduced.

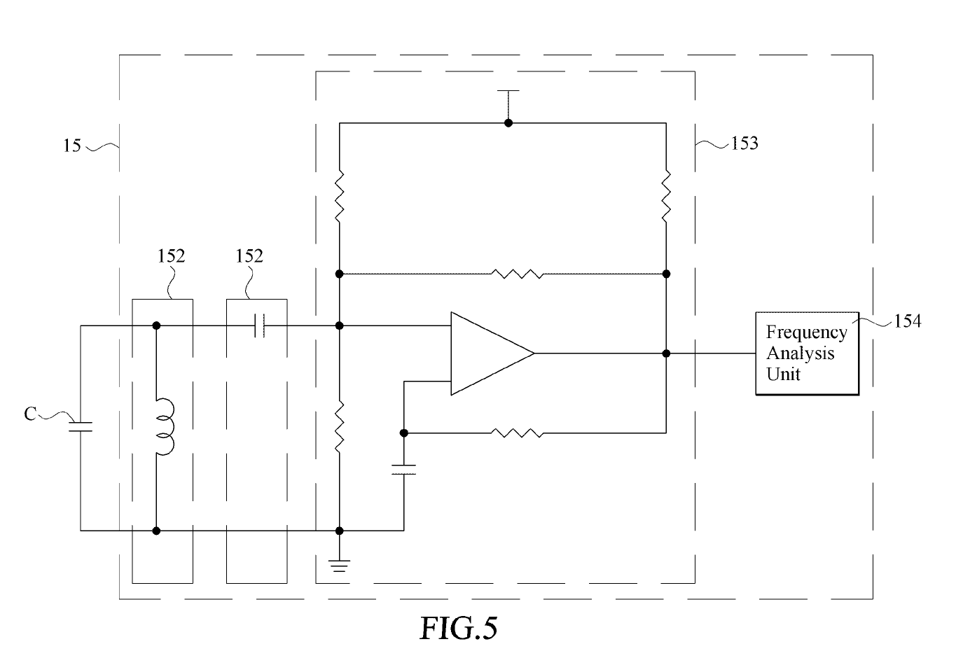

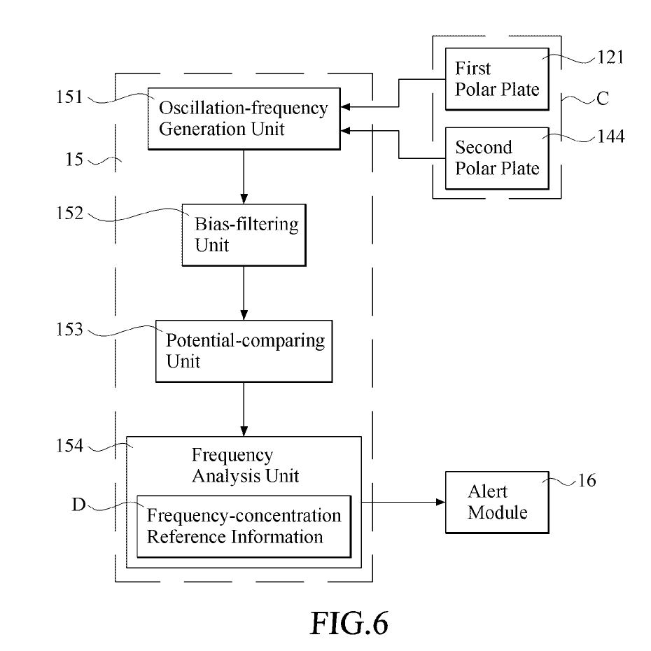

Refer now to FIG. 4, FIG. 5 and FIG. 6; where FIG. 5 is a schematic circuit view of the first embodiment of the frying apparatus capable of real-timely detecting a total polar material concentration of an oil in accordance with the present invention, and FIG. 6 is a schematic block view of this first embodiment. As shown, the detection module 15 further includes an oscillation-frequency generation unit 151, a bias-filtering unit 152, a potential-comparing unit 153 and a frequency analysis unit 154. The oscillation-frequency generation unit 151 electrically connected with each of the first polar plate 121 and the second polar plate 144 is to generate an oscillation signal while the equivalent capacitor is formed by the first polar plate 121 and the second polar plate 144 in the effective region AC for formulating the parallel capacitance effect.

Then, the bias-filtering unit 152 is applied to filter a DC (direct current) bias in the oscillation signal. The potential-comparing unit 153 is to transform the filtered oscillation signal into a non-offset AC (alternating current) signal. Thereupon, the oscillation signal can be analyzed to obtain a corresponding oscillation frequency for the oil. In this embodiment, the oscillation-frequency generation unit 151 can be a resonant inductor circuit, the bias-filtering unit 152 can be a bias-filtering capacitor circuit, and the potential-comparing unit 153 can be a comparator-amplifier interface circuit.

The frequency analysis unit 154 electrically connected with the potential-comparing unit 153 is stored with frequency-concentration reference information D. The frequency analysis unit 154 bases on the oscillation frequency to inquire a corresponding total polar material concentration of the oil from the frequency-concentration reference information D. In this embodiment, the frequency analysis unit 154 can be, but not limited to, a microcontroller unit (MCU).

In a typical experiment, a dielectric constant of the oil shall be corresponding to a total polar material concentration. Under the same coupling conditions, the dielectric constant of the oil is proportional to the equivalent capacitance of the oil. The oscillation-frequency generation unit 151 bases on the equivalent capacitance to generate an oscillation signal corresponding to the oscillation frequency of the oil. Thus, according to the oscillation frequency and the total polar material concentration of the oil corresponding to the oscillation signal, the related frequency-concentration reference information D can be obtained. Namely, the frequency analysis unit 154 can base on oscillation frequency of the oil corresponding to the received oscillation signal to locate the respective frequency-concentration reference information D, and therefrom the corresponding total polar material concentration can be obtained. For example, in the case that the inductance of the oscillation-frequency generation unit 151 is 1750 μH, then the related frequency-concentration reference information D would be listed as follows.

Table of the frequency-concentration reference information
(with an inductance of 1750 μH)

| | | | Total polar |
|---|---|---|---|
| | | | material |
| Equivalent | Dielectric | Resonance | concentration |
| capacitance (nF) | constant of oil | frequency (kHz) | (%) |
| 0.2 | 1.67 | 269 | 6 |
| 0.22 | 1.83 | 256.5 | 8 |
| 0.24 | 2.00 | 245.6 | 10 |
| 0.26 | 2.17 | 235.9 | 12 |
| 0.28 | 2.33 | 227.4 | 14 |
| 0.3 | 2.50 | 219.7 | 16 |
| 0.32 | 2.67 | 212.7 | 18 |
| 0.34 | 2.83 | 206.3 | 20 |
| 0.36 | 3.00 | 200.5 | 22 |
| 0.38 | 3.17 | 195.2 | 24 |
| 0.4 | 3.33 | 190.2 | 26 |

As shown in FIG. 3, the alert module 16 for alerting a message related to the total polar material concentration provided by the detection module 15 is communicatively connected with the frequency analysis unit 154 (referred to FIG. 5 and FIG. 6). In this embodiment, the alert module 16 can be a monitor for displaying directly the total polar material concentration. In the present invention, the alert module 16 can be, but not limited to, an indicator, buzzer or a display screen. The message related to the total polar material concentration can be, but not limited to, an indicator light or an alerting sound.

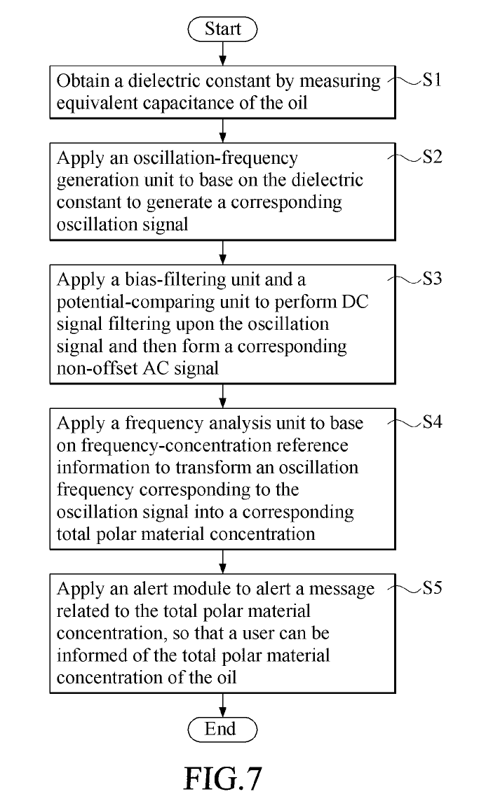

Refer now to FIG. 6 and FIG. 7, where FIG. 7 is a flowchart of a preferred detection method for the first embodiment of the frying apparatus to undergo a real-time detection of the total polar material concentration of a cooking oil in accordance with the present invention. As shown, firstly in Step S1, obtain a dielectric constant of the cooking oil by measuring an equivalent capacitance of the oil 2 (shown in FIG. 3). Further, in Step S2, apply an oscillation-frequency generation unit 151 to base on the dielectric constant of the oil to generate a corresponding oscillation signal. Then, in Step S3, apply a bias-filtering unit 152 and a potential-comparing unit 153 to perform DC signal filtering upon the oscillation signal so as to form a corresponding non-offset AC signal. Then, in Step S4, apply a frequency analysis unit 154 to base on frequency-concentration reference information D to transform an oscillation frequency corresponding to the oscillation signal into a corresponding total polar material concentration. Finally, in Step S5, apply an alert module 16 to alert a message related to the total polar material concentration, so that a user can be informed of the total polar material concentration of the oil.

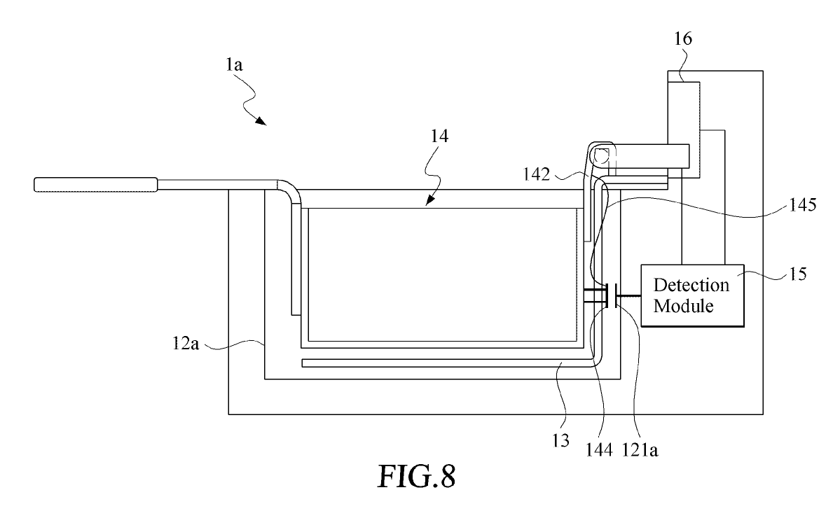

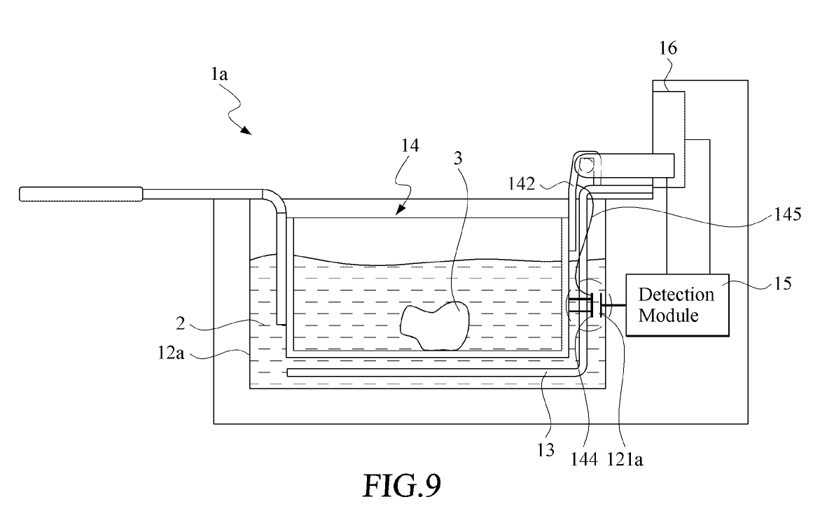

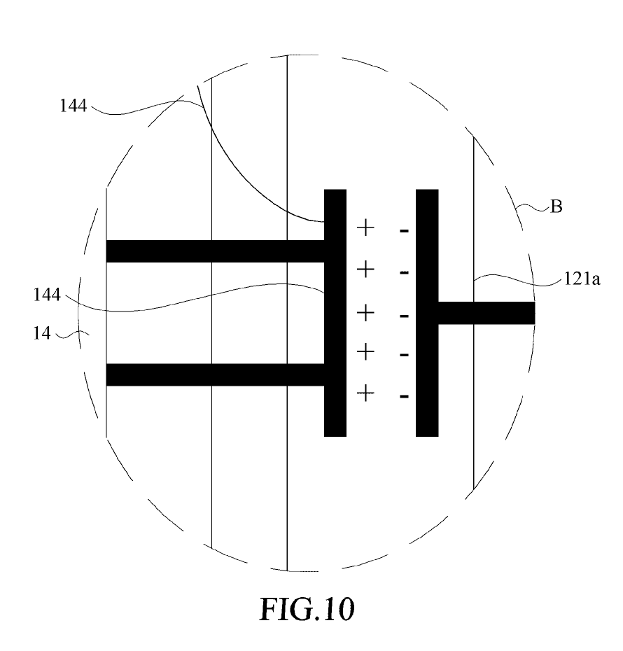

Refer now to FIG. 8, FIG. 9 and FIG. 10; where FIG. 8 is a schematic side view of a second embodiment of the frying apparatus capable of real-timely detecting a total polar material concentration of an oil in accordance with the present invention, FIG. 9 is an application of FIG. 8, and FIG. 10 is an enlarged view of area B of FIG. 9.

As shown, although the second embodiment 1a of the frying apparatus capable of real-timely detecting a total polar material concentration of an oil is similar structurally and electrically to the first embodiment 1 thereof in FIG. 2, yet one major difference in between is that, in this second embodiment 1a, the first polar plate 121a of the frying vat 12a is protruded from the inner wall of the frying vat 12a. In this embodiment, the first polar plate 121a is embodied as an isolated conductive plate, not a portion of the inner wall like the first embodiment does. In addition, the second conductive connector 142 is electrically connected to the second polar plate 144 by an electric wiring 145, unlike the first embodiment that the second conductive connector 142 is electrically connected to the second polar plate 144 through the conductive fry basket 14. Empirically, electric connection between the second conductive connector and the second polar plate through the electric wiring in the second embodiment can provide better conduction effect than that through the fry basket in the first embodiment.

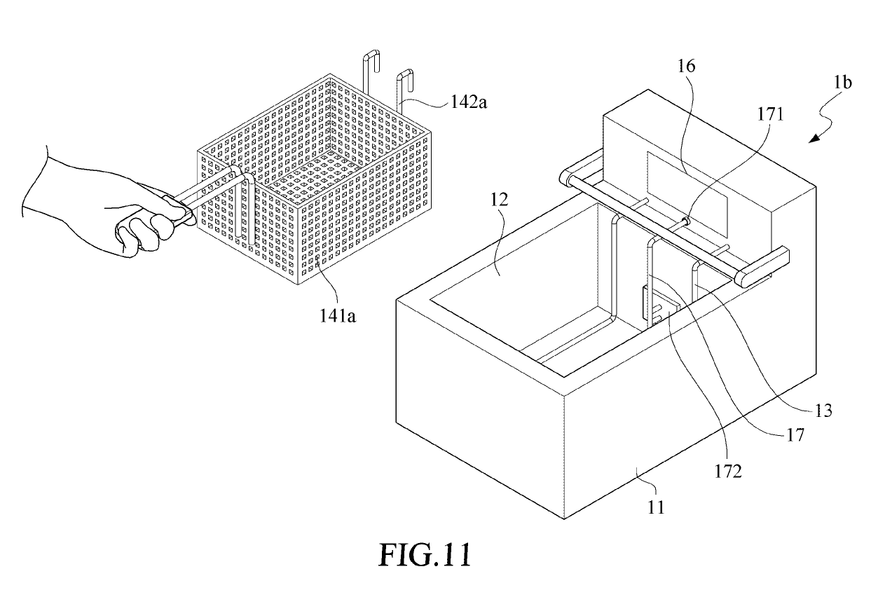

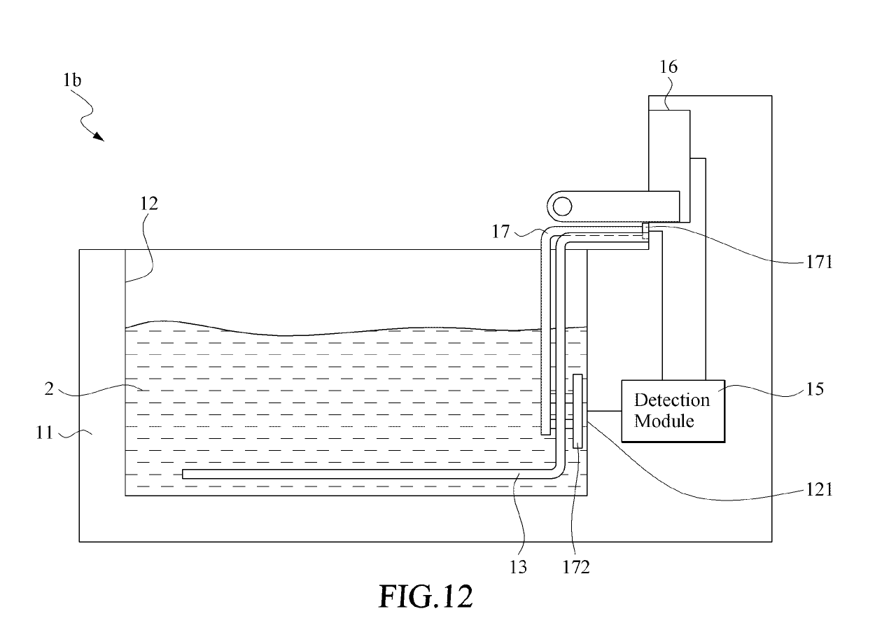

Refer now to FIG. 11 and FIG. 12; where FIG. 11 is a schematic perspective view of a third embodiment of the frying apparatus capable of real-timely detecting a total polar material concentration of an oil in accordance with the present invention, with the fry basket separated; and, FIG. 12 is a schematic side view of FIG. 11, without the fry basket, but with the cooking oil.

As shown, although the third embodiment 1b of the frying apparatus capable of real-timely detecting a total polar material concentration of an oil is similar structurally and electrically to the first embodiment 1 thereof in FIG. 1, yet one major difference in between is that, in this third embodiment 1b, the second polar plate 172 is a fixed structure without any structural and electric connection with the fry basket. Namely, the electric connection between the detection module and the second conductive connector in this third embodiment is not through the basket frame and the second conductive connector anymore like the first embodiment does. In this third embodiment 1b, the second polar plate 172 located inside the frying vat is electrically connected with, and also supported by, a polar-plate connection arm 17. The polar-plate connection arm 17 is extended out of the frying vat 12 from the second polar plate to 172. On the other end, the polar-plate connection arm 17 is electrically connected with the detection module 15 located at the detection module 15, such that the second polar plate 172 can be thus electrically connected with the detection module 15. Thereupon, while the second polar plate 172 is completely surrounded by the oil 2 in the frying vat 12, a close loop would be established by connecting electrically the first polar plate 121, the second polar plate 172, the polar-plate connection arm 17, the detection module 15 and finally the first polar plate 121 again, in which the oil 2 between the second polar plate 172 and the first polar plate 121 would act to formulate the capacitance effect.

In addition, one end of the polar-plate connection arm 17 that is connected with the main body 11 is further sleeved by an insulation ring 171. With this insulation ring 171, the polar-plate connection arm 17 can be insulated from the main body 11. In this embodiment, the polar-plate connection arm 17 is made of a conductive material, such as a metal, carbon or any the like. Further, the polar-plate connection arm 17 can be coated by an insulation layer or wrapped by an insulation material, so as to insulate the polar-plate connection arm 17 from the oil 2.

In summary, while the frying apparatus of the present invention is frying an object, the total polar material concentration of the oil can be detected simultaneously. In the first and second embodiment, when the oil fills the frying vat to form the effective region for formulating the parallel capacitance effect, an electric loop would be established by connecting electrically the first polar plate, the second polar plate, the second conductive connector, the first conductive connector and the detection module. In the third embodiment, when the oil enables the parallel capacitance effect in the effective region, the electric loop would be established by connecting electrically the first polar plate, the second polar plate, the polar-plate connection arm and the detection module. Upon these arrangements for the electric loop, the detection module can then detect the equivalent capacitance existing between the first polar plate and the second polar plate, and then this detected equivalent capacitance would be transformed into a corresponding dielectric constant of the oil.

Further, an oscillation-frequency generation unit is applied to transform a dielectric constant of the oil into a corresponding oscillation frequency of the oil. Then, a bias-filtering unit, a potential-comparing unit and a frequency analysis unit are applied orderly to transform the oscillation frequency of the oil into a corresponding total polar material concentration. Finally, an alert module is applied to alert a message related the total polar material concentration, so that a user of the frying apparatus can be aware of the instant total polar material concentration of the oil.

By compared with the prior art, the frying apparatus of the present invention can detect the instant total polar material concentration of the oil in a real-time manner, with an action time less than a second. In addition, since the dielectric property of the oil is used to deduce the total polar material concentration of the oil and the total polar material concentration of the oil is provided to be displayed by the alert module, thus the conventional shortcoming in judging the total polar material concentration by naked eyes can be totally resolved, and thereby possible visual bias caused by naked-eye observation can be effectively avoided. In addition, since the frying apparatus of the present invention can measure the total polar material concentration of the oil in a real-time manner, thus a huge expense to purchase additional instruments for detecting the total polar material concentration of the oil can be saved.

While the present invention has been particularly shown and described with reference to a preferred embodiment, it will be understood by those skilled in the art that various changes in form and detail may be without departing from the spirit and scope of the present invention.

What is claimed is:

1. A frying apparatus capable of real-timely detecting a total polar material concentration of an oil, comprising:
a main body;
a frying vat, located on the main body, being to contain an oil, further including a first polar plate;
a fry basket, further including:
a basket frame, to be placed into the frying vat; and
a second polar plate, connected with the basket frame; wherein, when the fry basket is positioned inside the frying vat, the second polar plate is parallel to and separated from the first polar plate so as to form an effective region for formulating a parallel capacitance effect between the first polar plate and the second polar plate; wherein the first polar plate and the second polar plate fulfill at least one coupling condition; and
a detection module, electrically connected with the first polar plate and the second polar plate;
wherein, when the first polar plate and the second polar plate are dipped in the oil contained inside the frying vat so as to have the oil to fill the effective region, the detection module detects an equivalent capacitance induced between the first polar plate and the second polar plate, a corresponding dielectric constant of the oil is then obtained by evaluating the equivalent capacitance and the at least one coupling condition, and further a corresponding total polar material concentration of the oil with respect to the dielectric constant is deduced.

2. The frying apparatus capable of real-timely detecting a total polar material concentration of an oil of claim 1, wherein the main body includes a first conductive connector electrically connected with the detection module, and the fry basket further includes a second conductive connector located at the basket frame, electrically connected with the second polar plate, and connected with the first conductive connector in a removable manner.

3. The frying apparatus capable of real-timely detecting a total polar material concentration of an oil of claim 2, further including an electric wiring to bridge electrically the second conductive connector and the second polar plate.

4. The frying apparatus capable of real-timely detecting a total polar material concentration of an oil of claim 2, wherein the first conductive connector is a conductive rod and the second conductive connector is a conductive hook.

5. The frying apparatus capable of real-timely detecting a total polar material concentration of an oil of claim 1, wherein the detection module is located at the main body.

6. The frying apparatus capable of real-timely detecting a total polar material concentration of an oil of claim 1, wherein the detection module further includes:
an oscillation-frequency generation unit, electrically connected with each of the first polar plate and the second polar plate, being to generate an oscillation signal when an equivalent capacitor in the effective region filled with the oil is formed by the first polar plate and the second polar plate, the oscillation signal being further analyzed to obtain a corresponding oscillation frequency of the oil; and
a frequency analysis unit, electrically connected with the oscillation-frequency generation unit, having frequency-concentration reference information; wherein, when the at least one coupling condition is satisfied, the oscillation-frequency generation unit bases on the oscillation frequency to inquire the corresponding total polar material concentration of the oil from the frequency-concentration reference information.

7. The frying apparatus capable of real-timely detecting a total polar material concentration of an oil of claim 1, wherein the first polar plate is one of an inner wall of the frying vat and a conductive plate.

8. The frying apparatus capable of real-timely detecting a total polar material concentration of an oil of claim 1, further including a heating member for heating the oil in the frying vat.

9. The frying apparatus capable of real-timely detecting a total polar material concentration of an oil of claim 1, further including an alert module communicatively connected with the detection module, being to alert a message related to the total polar material concentration.

10. The frying apparatus capable of real-timely detecting a total polar material concentration of an oil of claim 1, wherein the fry basket further includes a handle structure connected structurally with the basket frame; wherein, when the basket frame is positioned in the frying vat, the handle structure is located out of the frying vat from the basket frame.

11. A frying apparatus capable of real-timely detecting a total polar material concentration of an oil, comprising:
a main body;
a frying vat, located on the main body, being to contain an oil, further including a first polar plate;
a second polar plate, located in the frying vat at a position corresponding to the first polar plate so as to form an effective region for formulating a parallel capacitance effect between the first polar plate and the second polar plate; wherein the first polar plate and the second polar plate fulfill at least one coupling condition; and
a detection module, electrically connected with the first polar plate and the second polar plate;
wherein, when the first polar plate and the second polar plate are dipped in the oil contained inside the frying vat so as to have the oil to fill the effective region, the detection module detects an equivalent capacitance induced between the first polar plate and the second polar plate, a corresponding dielectric constant of the oil is then obtained by evaluating the equivalent capacitance and the at least one coupling condition, and further a corresponding total polar material concentration of the oil with respect to the dielectric constant is deduced.

12. The frying apparatus capable of real-timely detecting a total polar material concentration of an oil of claim 11, wherein the second polar plate is electrically connected the detection module via a polar-plate connection arm extending out of the frying vat from the second polar plate.
